(19) United States
(12) Patent Application Publication
(10) Pub. No.: US 2012/0316557 A1
(43) Pub. Date: Dec. 13, 2012

(54) Septoplasty Instrument

(75) Inventors: Joe D. Sartor, Longmont, CO (US); David N. Heard, Boulder, CO (US)

(73) Assignee: TYCO Healthcare Group LP, Boulder, CO (US)

(21) Appl. No.: 13/156,161

(22) Filed: Jun. 8, 2011

(51) Int. Cl. A61B 18/14

(52) U.S. Cl. 606/41

(57) ABSTRACT

A septoplasty instrument includes at least one shaft having an end effector at a distal end thereof. The end effector includes first and second electrode members, each having an electrode disposed within an insulative housing. At least one of the housings includes a lumen configured to circulate a cooling fluid therethrough. The lumen extends the length of the electrode member along an outer periphery thereof. At least one of the electrodes is adapted to conduct energy through tissue disposed therebetween. The electrode members are positionable on either side of the nasal septum. Upon application of energy to the electrode(s), energy is conduced between the electrodes and through the nasal septum such that the nasal septum is heated above about 50° C. and up to about 57° C. to allow reformation thereof, while the cooling fluid is circulated within the lumen to maintain tissue surrounding the electrode in a cooled state.

### BACKGROUND

The present disclosure relates to surgical instruments. More particularly, the present disclosure relates to a septoplasty instrument and method for correcting and/or straightening the nasal septum.

### TECHNICAL FIELD

The nasal septum is the wall between the nostrils that separates the two nasal passageways. The nasal septum is made partly of bone (the rear portion of the septum) and partly of cartilage (the front, or tip portion of the septum) and has three main functions: to support the nose, to regulate air flow, and to support the mucosa of the nose. The nasal septum, and particularly the cartilage portion of the nasal septum, may become crooked (deviated) or even dislocated as a result of aging or trauma to the nose, or may be a congenital condition.

A deviated septum is a relatively common condition among adults and generally does not require correction. However, in situations where the deviated or dislocated septum causes breathing difficulties, chronic sinusitis, snoring, sleep apnea, chronic nosebleeds and/or other problems, it may be necessary to correct the deviation or dislocation via a septoplasty, or septal reconstruction procedure.

Septoplasty is a surgical procedure for correcting, straightening and/or re-shaping the cartilage portion of the nasal septum. Typically, a septoplasty is performed under general or local anesthesia. The surgeon, working through the nostrils, makes an incision in the nasal mucosa, the soft tissue layer lining the nasal passages, to separate the mucosa from the septum. The surgeon then trims the extra length from the septum that was due to its deviation, repositions the septum, replaces and reattaches the mucosa over the septum, and stabilizes the septum, e.g., with small tubes, splints, or sutures. Often, after correcting the deviation of the septum, the surgeon will correct, or re-size the inferior and middle turbinates to conform the turbinates to the re-shaped nasal passageways.

### SUMMARY

A surgical instrument capable of performing a minimally-invasive in-office septoplasty is disclosed obviating the need for the splinting and suturing typically required to stabilize the septum and re-attach the mucosa. In particular, the shrinking of the collagen reduces the length of the septum eliminating the need to trim the length.

The present disclosure relates to a septoplasty instrument including one or more shafts having an end effector assembly disposed at a distal end thereof. The end effector assembly includes first and second electrode members spaced-apart from one another. Each electrode member includes an electrode disposed within an insulative housing. The housing of one (or both) of the electrodes includes a lumen defined therein configured to circulate a cooling fluid therethrough. The lumen extends a length of the electrode member along an outer periphery thereof. One (or both) of the electrodes is adapted to connect to a source of electrosurgical energy for conducting energy through tissue disposed between the electrode members. Each electrode member is also configured for insertion into a nostril of a patient such that the nasal septum of the patient is disposed between the first and second electrode members. Upon application of electrosurgical energy to one (or both) of the electrodes, energy is conducted between the electrodes and through the nasal septum such that the nasal septum is heated above about 50° C. and up to about 57° C. to allow reformation thereof. At the same time, the cooling fluid is circulated within the lumen to maintain tissue surrounding the electrode in a cooled state.

In one embodiment, a first electrical potential is provided to the first electrode member and a second electrical potential is provided to the second electrode member such that energy is conducted between the electrodes and through the nasal septum disposed therebetween.

In another embodiment, the cooling fluid is water. Alternatively, the cooling fluid may be glycol.

In another embodiment, first and second shaft members are provided, the first and second shaft members having the respective first and second electrode members disposed at distal ends thereof. The first and second shaft members may be pivotable with respect to one another about a pivot to move the electrode members between a spaced position relative to one another and a closer position relative to one another for grasping the nasal septum therebetween. Further, a ratchet mechanism may be provided for selectively locking the shaft members relative to one another.

In still another embodiment, the cooling fluid circulating through the lumen of the electrode member(s) maintains the tissue surrounding the electrode members below a predetermined temperature, e.g., below about 413° C.

The electrode members may define a paddle-shaped configuration. Alternatively, the electrode members may define a needle-shaped configuration having a sharpened distal end configured to partially (or entirely) penetrate surface tissue adjacent the nasal septum.

In accordance with another embodiment of the present disclosure, a septoplasty instrument is provided. The septoplasty instrument includes one (or more) shafts having an end effector assembly disposed at distal end thereof. One of the shafts is adapted to connect to a fluid source for supplying cooling fluid to the end effector assembly. The end effector assembly includes first and second electrode members spaced-apart from one another. Each electrode member includes an electrode disposed within an insulative housing. The housing of one (or both) of the electrodes includes a lumen defined therein configured to circulate a cooling fluid therethrough. The lumen has an input and an output and extends a length of the electrode member along an outer periphery thereof. The lumen is configured for circulating a cooling fluid from the shaft connected to the fluid source, into the input, through the lumen, and out of the output. One (or both) of the electrodes is adapted to connect to a source of electrosurgical energy for conducting energy through tissue disposed therebetween. Each electrode member is configured for insertion into a nostril of a patient such that the nasal septum of the patient is disposed between the first and second electrode members. Upon application of electrosurgical energy to the electrode(s), energy is conducted between the electrodes and through the nasal septum such that the nasal septum is heated above about 50° C. and up to about 57° C. to allow reformation thereof. At the same time, the cooling fluid is circulated within the lumen to maintain tissue in immediate contact with the electrode in a cooled state.

In embodiments, a temperature sensor is located on the surface of one (or both) of the electrode members to monitor mucosal surface temperature. The temperature sensor may further be configured to regulate power provided to the electrode member(s) to assure mucosal tissue does not exceed the maximum temperature, e.g., about 45° C., or, preferably, about 40° C.

A method of performing a septoplasty is also provided in accordance with the present disclosure. The method includes providing first and second electrode members, each including an electrode disposed within an insulative housing. The housing of one (or both) of the electrodes includes a lumen defined therein configured to circulate a cooling fluid therethrough. The lumen extends a length of the electrode member along an outer periphery thereof. One (or both) of the electrodes is adapted to connect to a source of electrosurgical energy for conducting energy through tissue disposed therebetween. Upon application of electrosurgical energy to the electrode(s), energy is conducted between the electrodes and through the nasal septum such that the nasal septum is heated. The method further includes the steps of inserting the first electrode member into a first nostril of a patient, inserting the second electrode member into a second nostril of the patient such that the nasal septum is grasped between the first and second electrode members, allowing the mucosa to cool below body temperature, conducting electrosurgical energy between the first and second electrode members and through the nasal septum to heat the nasal septum above about 50° C. and up to about 57° C. to allow reformation thereof while circulating the cooling fluid within the lumen to maintain tissue surrounding the electrode in a cooled state, and reforming the nasal septum to a reformed configuration.

In one embodiment, the method further includes the steps of maintaining the nasal septum in the reformed configuration and allowing the nasal septum to cool such that the nasal septum retains the reformed configuration.

Also provided in accordance with the present disclosure is a septoplasty instrument and system wherein the source of electrosurgical energy monitors the impedance through the electrode members such that, as conductivity increases following heating of the septum to about 50° C., power supplied to the electrode members is decreased, or controlled, to prevent overheating of the septal tissue.

### BRIEF DESCRIPTION OF THE DRAWINGS

Various embodiments of the presently disclosed instrument are described herein with reference to the drawings, wherein:

FIG. 1 is a front, perspective view of a septoplasty instrument in accordance with one embodiment of the present disclosure;

FIG. 2 is a side, cross-sectional view of one of the shaft members of the septoplasty instrument of FIG. 1;

FIG. 3 is an enlarged, perspective view of the area of detail of FIG. 1 showing an end effector assembly configured for use with the septoplasty instrument;

FIG. 4A is an enlarged, perspective view of one of the paddle members of the end effector assembly of FIG. 3;

FIG. 4B is an enlarged, perspective view of the other paddle member of the end effector assembly of FIG. 3;

FIG. 5 is an enlarged, perspective view of another embodiment of an end effector assembly configured for use with the septoplasty instrument of FIG. 1;

FIG. 6A is an enlarged, perspective view of one of the needle members of the end effector assembly of FIG. 5;

FIG. 6B is an enlarged, perspective view of the other needle member of the end effector assembly of FIG. 5;

FIG. 7 is a schematic illustration of a deviated septum;

FIG. 8 is a schematic illustration of the septum of FIG. 7 after a septoplasty procedure in accordance with the present disclosure has been performed; and

FIG. 9 is a graphical representation indicating the conductivity of cartilage tissue as a function of temperature.

### DETAILED DESCRIPTION

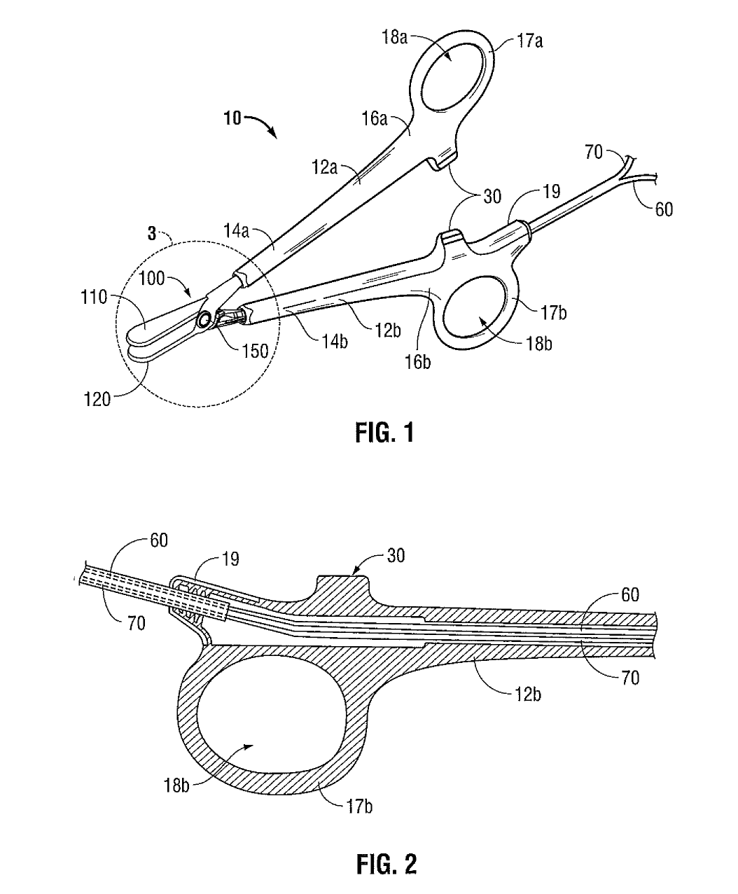

Referring initially to FIG. 1, a septoplasty instrument 10 includes two elongated shafts 12a and 12b each having a proximal end 16a and 16b and a distal end 14a and 14b, respectively. In the drawings and in the descriptions which follow, the term “proximal,” as is traditional, will refer to the end of the septoplasty instrument 10 that is closer to the user, while the term “distal” will refer to the end that is further from the user.

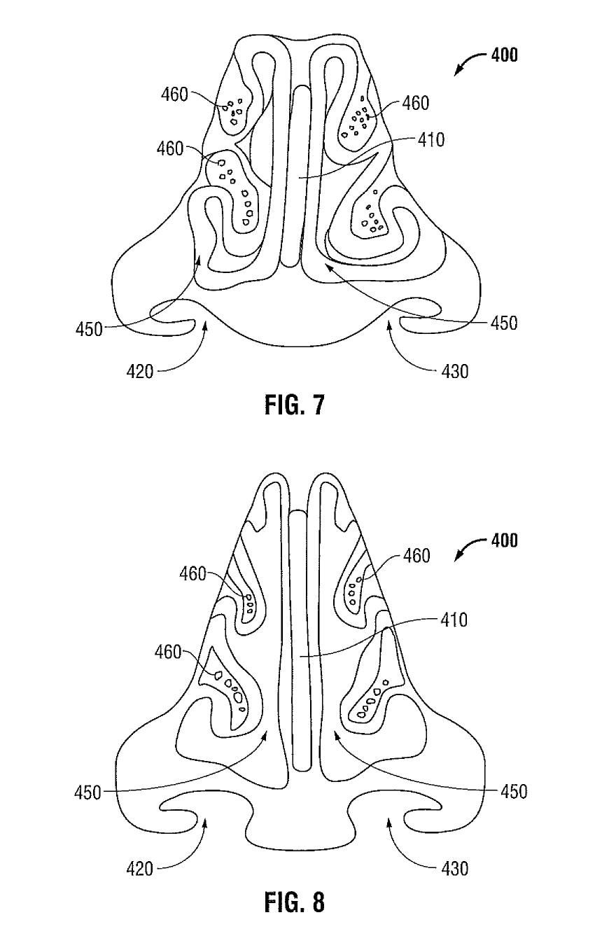

Septoplasty instrument 10 includes an end effector assembly 100 attached to distal ends 14a and 14b of shafts 12a and 12b, respectively. As explained in more detail below, end effector assembly 100 includes a pair of opposing electrode members 110 and 120 that are pivotably connected about a pivot pin 150. Septoplasty instrument 10 is configured for insertion into the nostrils 420, 430 (FIGS. 7 and 8) of a patient such that the nasal septum 410 (FIGS. 7 and 8) may be grasped between opposing electrode members 110, 120.

Each shaft 12a and 12b includes a handle 17a and 17b disposed at a proximal end 16a and 16b, respectively, thereof. Each handle 17a and 17b defines a finger hole 18a and 18b, respectively, therethrough for receiving a finger of the user. As can be appreciated, finger holes 18a and 18b facilitate movement of shafts 12a and 12b relative to one another which, in turn, pivots electrode members 110 and 120 from a spaced position, wherein electrode members 110 and 120 are disposed in spaced-apart relation relative to one another to a closer position (FIG. 1), wherein electrode members 110 and 120 cooperate to grasp the nasal septum 400 (see FIG. 7) therebetween.

Depending on the anatomy of the patient's nose 400 (FIGS. 7 and 8), electrode members 110 and 120 may be disposed in different positions relative to one another when grasping the nasal septum 410 (FIGS. 7 and 8) of the patient between electrode members 110 and 120. Accordingly, a ratchet 30 may be included for selectively locking the electrode members 110 and 120 relative to one another at various positions during pivoting. Ratchet 30 may include graduations or other visual markings that enable the user to easily and quickly ascertain and control the spacing between electrode members 110 and 120 and the amount of closure force desired between electrode members 110 and 120 to adapt septoplasty instrument 10 to the specific anatomy of the patient.

With reference to FIGS. 1 and 2, one of the shafts, e.g., shaft 12b, includes a proximal shaft connector 19 that is designed to connect the septoplasty instrument 10 to a source of electrosurgical energy such as an electrosurgical generator (not shown). Proximal shaft connector 19 secures an electrosurgical cable 60 to septoplasty instrument 10 such that the user may selectively apply electrosurgical energy from the generator (not shown) to either (or both) of electrode members 110, 120. Proximal shaft connector 19 may also be configured to secure a fluid cable, or cables 70 to shaft 12b such that fluid may be circulated through electrode members 110, 120 to maintain the outer peripheral surfaces 113, 123, of electrode members 110, 120, respectively, in a relatively cooled state. Although proximal shaft connector 19 is shown connected to shaft 12b, either (or both) of shafts 12a and 12b may be configured to secure electrosurgical cable 60 and/or fluid cable(s) 70 thereto.

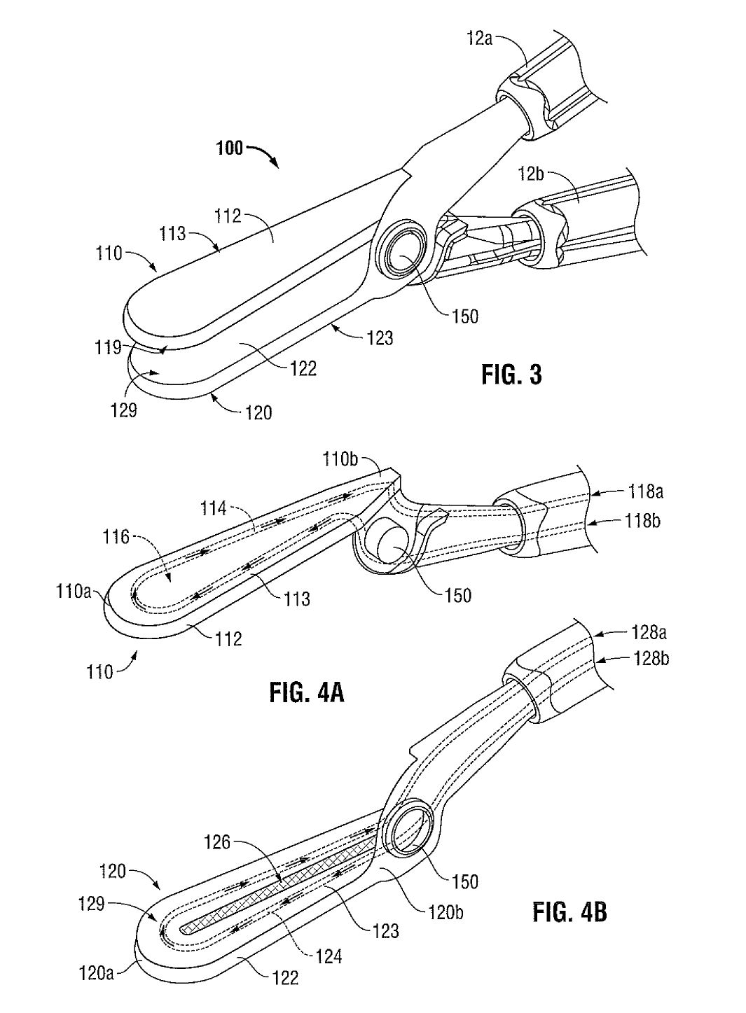

As mentioned above, the two opposing electrode members 110 and 120 of end effector assembly 100 are pivotable about pivot pin 150 from the spaced position to the closer position for grasping the nasal septum 410 (FIGS. 7 and 8) therebetween. As shown in FIG. 3, electrode members 110 and 120 of end effector assembly 100 may each define a generally paddle-shaped configuration to facilitate grasping of the nasal septum 410 (FIGS. 7 and 8) therebetween. Each electrode member 110, 120 of end effector assembly 100 may be configured for insertion into a respective nostril 420, 430 (FIGS. 7 and 8) of a patient such that electrode members 110, 120 are positionable on opposite sides of and adjacent to the nasal septum 410 (FIGS. 7 and 8) along a substantial length of the nasal septum 410 (FIGS. 7 and 8).

Turning now to FIGS. 4A and 4B, in conjunction with FIG. 3, end effector assembly 100 will be described in greater detail. Electrode members 110, 120, respectively, of end effector assembly 100 each define a paddle-shaped configuration and include an outer, insulative housing 112, 122 that houses a fluid lumen 114, 124 defined therein and an electrode 116, 126. Electrodes 116, 126 of respective electrode members 110, 120 extend longitudinally through housings 112, 122, respectively, along a substantial length thereof and may be centrally disposed therethrough. Electrode 116 and/or electrode 126 are coupled to electrosurgical cable 60, which, as shown in FIG. 2, extends through shaft 12b, ultimately connecting to a source of electrosurgical energy (not shown) for providing energy to electrode 116 and/or electrode 126. Fluid lumens 114, 124 surround electrodes 116, 126, respectively and create a fluid flow path from respective proximal ends 110b, 120b of electrode members 110, 120 to respective distal ends 110a, 120a of electrode members 110, 120 and returning from respective distal ends 110a, 120a of electrode members 110, 120 back to respective proximal ends 110b, 120b of electrode members 110, 120 such that fluid may be circulated through a substantial area of electrode members 110, 120. Fluid lumens 114, 124 may each further include a respective input tube 118a, 128b and a respective output tube 118b, 128b for directing fluid into and out of, respectively, electrode members 110, 120. Input and output tubes 118a, 118b of electrode member 110 and/or input and output tubes 128a, 128b of electrode member 120 are coupled to fluid cable(s) 70 which, as shown in FIG. 2, extends through shaft 12b, ultimately connecting to a fluid source (not shown) for supplying coolant fluid to electrode member 110 and/or electrode member 120. As will be described in greater detail below, the circulating coolant fluid is configured to maintain outer peripheral surfaces 113, 123 of electrode members 110, 120, respectively, in a cooled state while electrosurgical energy is conducted between electrodes 116, 126 of electrode members 110, 120, respectively, to heat the nasal septum 410 (FIGS. 7 and 8).

Insulative housings 112, 122 of respective electrode members 110, 120 of end effector assembly 100 may include more insulation surrounding the outer-facing surfaces and sides of paddle-shaped insulative housings 112, 122 than on respective opposed surfaces 119, 129, of housings 112, 122 to facilitate heating of the nasal septum 410 (FIGS. 7 and 8) while reducing the potential for thermal spread to surrounding tissue.

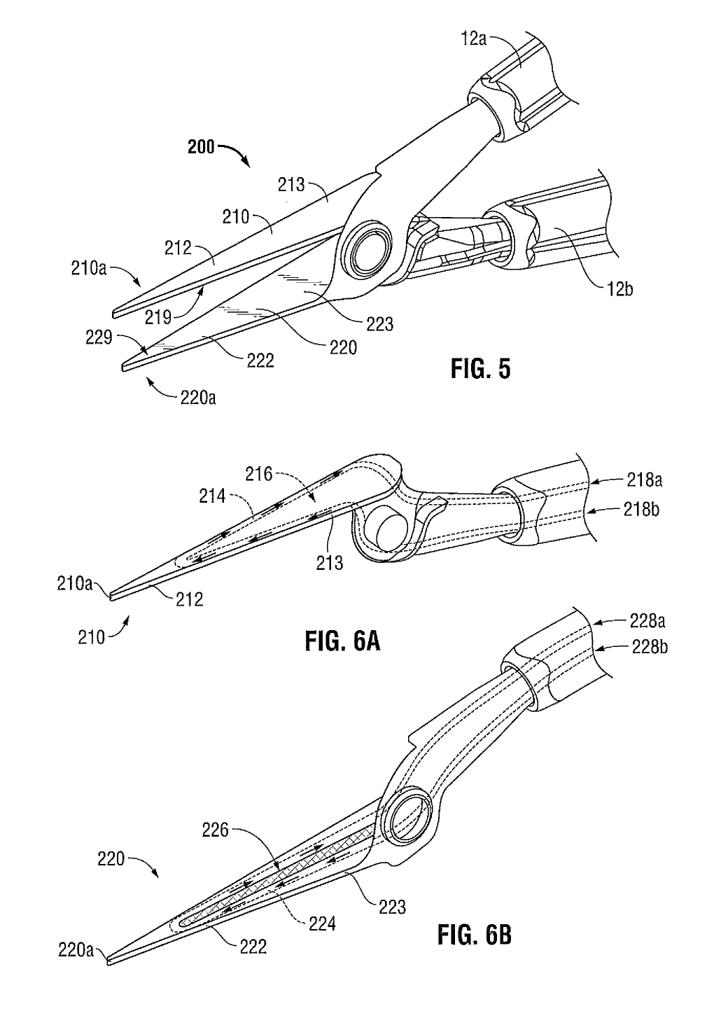

Another embodiment of an end effector assembly configured for use with septoplasty instrument 10, end effector assembly 200, is shown in FIG. 5. Each electrode member 210, 220 of end effector assembly 200 defines a needle-shaped configuration having a respective pointed distal tip 210a, 220a configured to penetrate the mucosa adjacent the nasal septum 410 (FIGS. 7 and 8) such that electrode members 210, 220 may be positioned in closer proximity to the nasal septum 410 (FIGS. 7 and 8).

Turning now to FIGS. 6A and 6B, in conjunction with FIG. 5, end effector assembly 200 will be described in greater detail. Electrode members 210, 220 of end effector assembly 200 each define a needle-shaped configuration including a pointed distal tip 210a, 220a. As with electrode members 110, 120, electrode members 210, 220 each include an outer, insulative housing 212, 222 that houses a coolant fluid lumen 214, 224 defined therein and an electrode 216, 226. Electrodes 216, 226 of respective electrode members 210, 220 extend longitudinally through housings 212, 222, respectively. One or both of electrode 216, 226 may be coupled to electrosurgical cable 60 for providing energy to electrode 216 and/or electrode 226, Fluid lumens 214, 224 surround electrodes 216, 226, respectively, and create a coolant fluid flow path around electrodes 216, 226, respectively. Input tubes 218a, 228a and output tubes 218b, 228b of fluid lumens 214, 224 of electrode members 210, 220, respectively, are coupled to fluid cable(s) 70 which, as shown in FIG. 2, extends through shaft 12b, for supplying fluid to electrode members 210, 220. As mentioned above, the circulating fluid maintains outer peripheral surfaces 213, 223 of electrode members 210, 220 in a cooled state when energy is conducted between electrode members 210, 220, to heat the nasal septum 410 (FIGS. 7 and 8).

Electrode members 210, 220, as mentioned above, include pointed distal tips 210a, 220a, respectively, that are configured to penetrate the mucosa such that electrode members 210, 220 may be positioned in direct contact with the nasal septum 410 (FIGS. 7 and 8). In such an embodiment, opposed surfaces 219, 229 of respective electrode members 210, 220 may be formed of a conductive material or may have reduced insulation as compared to the remainder of insulative housings 212, 222, such that energy is more easily conducted between electrodes 216, 226 of respective electrode members 210, 220 to heat the nasal septum 410 (FIGS. 7 and 8) while not advancing thermal energy to surrounding tissue. Further, fluid lumens 214, 224 may be configured to direct more cooling fluid to other portions of insulative housings 212, 222, respectively, rather than to respective opposing surfaces 219, 229, to further facilitate heating of the nasal septum 410 (FIGS. 7 and 8) while reducing the potential for thermal spread to surrounding tissue.

The fluid supplied from the fluid source (not shown) through fluid cable(s) 70 and circulated within the fluid lumens 114, 124 and 214, 224 of electrode members 110, 120 and 210, 220 of end effector assemblies 100, 200, respectively, may be water, glycol, or any other suitable non-conductive fluid that helps maintain outer peripheral surfaces 113, 123 and 213, 223 of respective electrode members 110, 120 and 210, 220 of end effector assemblies 100, 200, respectively, in a relatively cooled state to reduce thermal spread. More specifically, the circulation of fluid through the fluid lumens 114, 124, and 214, 224 may be configured to maintain respective outer peripheral surfaces 113, 123 and 213, 223 of electrode members 110, 120 and 210, 220, respectively, at a temperature at or below 45° C. and, in some embodiments, below about 40° C., such that tissue surrounding the nasal septum 410 (FIGS. 7 and 8), e.g., mucosa tissue, is substantially undamaged during heating of the nasal septum 410 (FIGS. 7 and 8).

The electrodes 116, 126 of respective electrode members 110, 120 of end effector assembly 100 may be configured as bipolar RF electrodes, In other words, a first electrical potential may be provided to electrode 116 and a second electrical potential may be provided to electrode 126 such that an electrical potential gradient is created for conducting RF energy between the electrodes 116, 126 and through the nasal septum 410 (FIGS. 7 and 8) disposed therebetween. Electrodes 216, 226 of electrode members 210, 220 of end effector assembly 200 may similarly be configured as bipolar RF electrodes. As will be described in greater detail below, the electrodes 116, 126 of end effector assembly 100 may be configured to heat the nasal septum 410 (FIGS. 7 and 8) to above about 50° C., wherein the nasal septum is heated to a partially relaxed temperature and up to about 57° C., the target relaxation temperature for nasal cartilage tissue. Within this temperature range, the cartilage of the nasal septum 410 (FIGS. 7 and 8) shrinks and becomes reformable. Electrodes 216, 226 of end effector assembly 200 may similarly be configured to heat the nasal septum 410 (FIGS. 7 and 8) above about 50° C. and up to about 57° C.

With reference to FIGS. 1-8, the operation of septoplasty instrument 10 will be described. Septoplasty procedures are most commonly preformed to re-shape, or straighten the nasal septum 410 in order to alleviate sinus, breathing, or other problems or simply for cosmetic reasons. FIG. 7 is a schematic illustration of the nose 400 of a patient who may benefit from a septoplasty procedure. As shown in FIG. 7, the nasal septum 410 is crooked, reducing the nasal passageways 450 on either side thereof. As can be appreciated, the goal of the septoplasty procedures described below with reference to end effector assemblies 100 and 200 of septoplasty instrument 10 is to reshape the nasal septum 410 to alleviate the above-mentioned problems.

Regarding the operation of end effector assembly 100 of septoplasty instrument 10, in preparation for insertion into the nostrils 420, 430 of a patient, septoplasty instrument 10 is moved to the spaced-apart position wherein electrode members 110, 120 are spaced-apart from one another. More specifically, electrode members 110, 120 are spaced-apart a sufficient distance such that each electrode member 110, 120 may be inserted into the nostrils 420, 430, respectively, of the patient.

With electrode members 110, 120 in the spaced position, as mentioned above, septoplasty instrument 10 is advanced into the nostrils 420, 430 such that electrode members 110, 120 are each disposed within one of the nostrils 420, 430, respectively, with the nasal septum 410 therebetween. Next, handles 17a, 17b, are squeezed toward one another to move electrode members 110, 120 to the closer position for grasping the nasal septum 410 therebetween. As the nasal septum 410 is grasped between electrode members 110, 120, the nasal septum 410 is deformed (although not permanently deformed at this point) to a straight configuration. In other words, grasping the nasal septum 410 between electrode members 110, 120 retains the cartilage of the nasal septum 410 in a straightened position. Ratchet 30 may be used to fix the relative position of electrode members 110, 120 to ensure a consistent and effective grasping of the nasal septum 410. As can be appreciated, in this position, paddle-shaped electrode members 110, 120 extend along a substantial length of the nasal septum 410 and cover a substantial area of the nasal septum 410.

Once septoplasty instrument 10 is properly positioned grasping the nasal septum 410 therebetween in a straightened position, energy, e.g., RF electrosurgical energy, may be applied to electrode 116 and/or electrode 126. As mentioned above, energy is supplied from an energy source, e.g., an electrosurgical generator (not shown), via electrosurgical cable 60, through one of the shaft members, e.g., shaft 12b, and to electrode 116 and/or electrode 126. Due to the electrical potential gradient between electrodes 116, 126, energy is conducted therebetween and, thus, through the nasal septum 410 to heat the nasal septum 410.

As mentioned above, energy is conducted through the nasal septum 410 to heat the nasal septum 410 above about 50° C. and up to about 57° C. At about 50° C., wherein the nasal cartilage tissue is in a partially relaxed state, the nasal septum 410 is softened and becomes reformable. At about 57° C., the target relaxation temperature for nasal cartilage tissue, the septum is fully reformable. As shown in FIGS. 4A-4B, a monitoring sensor(s) 180, e.g., a temperature or conductivity sensor, may be provided on the surface of one (or both) of electrode members 110, 120 to monitor mucosal surface temperature or properties of the nasal cartilage tissue, e.g., conductivity. Monitoring sensor(s) 180 may further be configured to regulate power provided to the electrode member(s) 110, 120 to assure mucosal tissue does not exceed the maximum temperature, e.g., about 45° C., or, preferably, about 40° C., and/or to maintain a desired conductivity of the nasal cartilage. The monitoring sensor 180 and/or other monitoring mechanisms (not shown) may also be used to control shrinkage of the nasal septum 410, which may result from increased heating of the nasal septum 410.

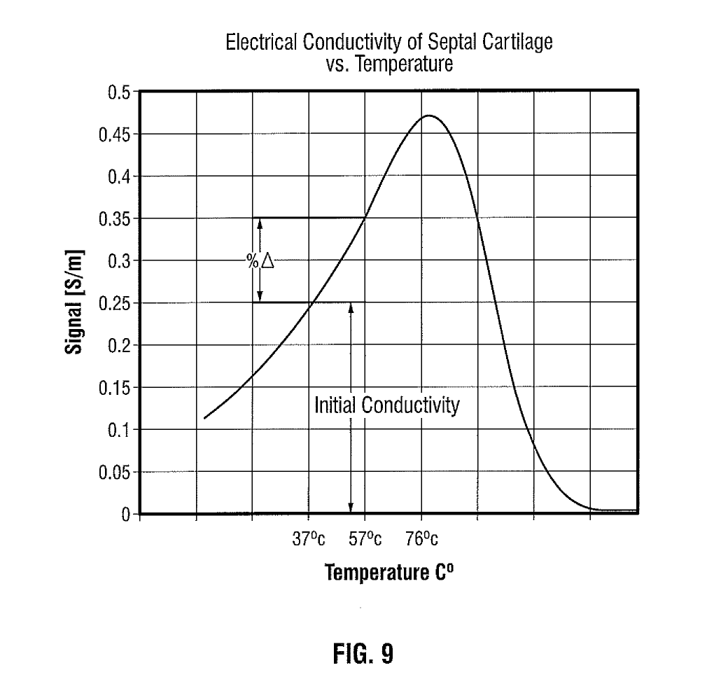

More specifically, with reference to FIG. 9, in conjunction with FIGS. 4A-4B, monitoring sensors 180 may be configured to monitor the electrical conductivity of the nasal cartilage tissue to achieve a target cartilage relaxation temperature of about 57 degrees C. In use, prior to the application of energy to the cartilage, i.e., when the cartilage is at normal body temperature (about 37 degrees C.), an initial conductivity of the cartilage is measured. The conductivity of the cartilage is incrementally or continuously measured thereafter as energy is supplied to electrodes 116, 126 to heat the nasal cartilage tissue. Once the relative percentage change in conductivity, % A, reaches approximately 40%, or on the order thereof, monitoring sensor 180 controls the application of energy to electrodes 116, 126 to maintain the conductivity at this target relative value, which corresponds to the target cartilage relaxation temperature of about 57 degrees C. For example, a closed loop control (of 1-5 seconds) may be used to maintain this target relative change in conductivity.

At the same time that electrosurgical energy is supplied to electrode 116 of electrode member 110 and/or to electrode 126 of electrode member 120, the fluid source (not shown) is activated to supply coolant fluid through fluid cable(s) 70 and into the fluid lumens 114, 124 of respective electrode members 110, 120 for maintaining the outer peripheral surfaces 113, 123 of respective electrode members 110, 120 in a relatively cooled state. Maintaining the outer peripheral surfaces 113, 123 of electrode members 110, 120, respectively, in a cooled state helps prevent substantial thermal damage to surrounding tissue. The circulation of fluid through fluid lumens 114, 124 may be configured to maintain surrounding tissue below about 45° C. and, more particularly, below about 40° C., during heating of the nasal septum 410.

In other words, the nasal septum 410 is heated by the conduction of energy between electrode 116 of electrode member 110 and electrode 126 of electrode member 120, while surrounding tissue is maintained in a cooled state by the circulation of coolant, or cooling fluid, through the fluid lumens 114, 124 of respective electrode members 110, 120. When the nasal septum 410 reaches a reformable state, e.g., when the nasal septum 410 is heated to about 75 degrees C., septoplasty instrument 10 may be repositioned to ensure proper alignment of the nasal septum 410. Once the nasal septum 410 is properly aligned, the supply of electrosurgical energy to electrodes 116, 126 is stopped, or cut-off, i.e., the nasal septum 410 is no longer actively heated, and, thus, the nasal septum 410 is allowed to cool. The flow of coolant fluid through the fluid lumens 114, 124 of electrode members 110, 120, respectively, may continue during the cooling process, to facilitate cooling of the nasal septum 410.

As the nasal septum 410 cools, it retains the reformed, e.g., straightened, shape. Once the nasal septum 410 has fully cooled back to body temperature, ratchet 30 may be disengaged, electrode members 110, 120 may be moved back to the spaced-apart position to release the grasp on the nasal septum 410, and septoplasty instrument 10 may be removed from the patient's nostrils 420, 430. FIG. 8 shows a reshaped nasal septum 410 such as, for example, the nasal septum 410 after the septoplasty procedure discussed above. As shown in FIG. 8, the nasal passageways 450 are no longer obstructed.

The operation of end effector assembly 200 of septoplasty instrument 10 is similar to the operation of end effector assembly 100. Initially, septoplasty instrument 10 is moved to the spaced-apart position wherein electrode members 210, 220 are spaced-apart from one another. Next, electrode members 210, 220 are inserted into the nostrils 420, 430 of the patient. Pointed distal tips 210a, 220a of electrode members 210, 220, respectively, are used to puncture the mucousa, forming a small incision therein to allow electrode members 210, 220 to be positioned directly adjacent the nasal septum 410 on either side thereof. Handles 17a, 17b of septoplasty instrument 10 are then squeezed toward one another to move electrode members 210, 220 to the closer position for grasping the nasal septum 410 therebetween.

With the nasal septum 410 grasped between electrode members 210, 220 of end effector assembly 200, as with the operation of end effector assembly 100, electrosurgical energy is supplied to electrode 216 and/or electrode 226 to heat the nasal septum 410. Similarly as mentioned above, sensors or other monitoring mechanisms may be provided for monitoring the temperature of the mucosal tissue, the conductivity of the nasal cartilage, or other properties of tissue to control the heating of electrode members 210, 220. At the same time as heating, coolant fluid is circulated through fluid lumens 214, 224 of electrode members 210, 220, respectively, to maintain surrounding tissue in a cooled state.

The nasal septum 410 is heated, as discussed above in relation to end effector assembly 100, until the nasal septum 410 is reformable to a straightened configuration. The nasal septum 410 is then allowed to cool, permanently reforming in the straightened configuration as shown, for example, in FIG. 8. Septoplasty instrument 10 may then be removed from the nostrils 420, 430 of the patient.

The electrode members 110, 120 and 210, 220 of end effector assembly 100 and/or end effector assembly 200, respectively, may be coated with a conductive gel, saline solution, or other suitable substance (not explicitly shown) to help prevent the tissue from sticking to electrode members 110, 120 and 210, 220. Further, other suitable cooling mechanisms, e.g., an electrical cooling mechanism (not shown) or a heat pipe mechanism (not shown), may be provided for maintaining electrode members 110, 120 and 210, 220 in a cooled state during heating of the nasal septum 410.

Although the above-mentioned procedure is mentioned with respect to a septoplasty, it is also envisioned that septoplasty instrument 10 may be used to reshape the turbinates 460 (as part of the septoplasty procedure or as an independent procedure), in order to alleviate other complications within the nasal passageways 450.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed:

1. A septoplasty instrument, comprising:
at least one shaft, the at least one shaft having an end effector assembly disposed at a distal end thereof, the end effector assembly including:
first and second electrode members spaced-apart from one another, each electrode member including an electrode disposed within an insulative housing, the housing of at least one electrode including a lumen defined therein configured to circulate a cooling fluid therethrough, the lumen extending a length of the electrode member along an outer periphery thereof, at least one of the electrodes adapted to connect to a source of electrosurgical energy for conducting energy through tissue disposed therebetween, each electrode member configured for insertion into a nostril of a patient such that the nasal septum of the patient is disposed between the first and second electrode members, wherein, upon application of electrosurgical energy to the at least one electrode, energy is conducted between the electrodes and through the nasal septum such that the nasal septum is heated above about 50° C. and up to about 57° C. to allow reformation thereof, and wherein the cooling fluid is circulated within the lumen to maintain tissue surrounding the electrode in a cooled state.

2. The septoplasty instrument according to claim 1, wherein a first electrical potential is provided to the first electrode member and wherein a second electrical potential is provided to the second electrode member such that energy is conducted between the electrodes and through the nasal septum disposed therebetween.

3. The septoplasty instrument according to claim 1, wherein the cooling fluid is one of water and glycol.

4. The septoplasty instrument according to claim 1, further comprising first and second shaft members having the respective first and second electrode members disposed at distal ends thereof.

5. The septoplasty instrument according to claim 4, wherein the first and second shaft members are pivotable with respect to one another about a pivot to move the electrode members between a spaced position relative to one another and a closer position relative to one another for grasping the nasal septum therebetween.

6. The septoplasty instrument according to claim 5, further comprising a ratchet mechanism configured for selectively locking the shaft members relative to one another.

7. The septoplasty instrument according to claim 1, wherein the cooling fluid circulating through the lumen of the at least one electrode members maintains the tissue surrounding the electrode members below a pre-determined temperature.

8. The septoplasty instrument according to claim 7, wherein the pre-determined temperatures is less than about 40° C.

9. The septoplasty instrument according to claim 1, wherein the electrode members define a paddle-shaped configuration.

10. The septoplasty instrument according to claim 1, wherein the electrode members define a needle-shaped configuration having a sharpened distal end configured to at least partially penetrate surface tissue adjacent the nasal septum.

11. A septoplasty instrument, comprising:
at least one shaft, the at least one shaft having an end effector assembly disposed at distal end thereof, one of the at least one shafts adapted to connect to a fluid source for supplying cooling fluid to the end effector assembly, the end effector assembly including:
first and second electrode members spaced-apart from one another, each electrode member including an electrode disposed within an insulative housing, the housing of at least one electrode including a lumen defined therein configured to circulate a cooling fluid therethrough, the lumen having an input and an output, the lumen extending a length of the electrode member along an outer periphery thereof and configured for circulating a cooling fluid from the shaft connected to the fluid source, into the input, through the lumen, and out of the output, at least one of the electrodes adapted to connect to a source of electrosurgical energy for conducting energy through tissue disposed therebetween, each electrode member configured for insertion into a nostril of a patient such that the nasal septum of the patient is disposed between the first and second electrode members, wherein, upon application of electrosurgical energy to the at least one electrode, energy is conducted between the electrodes and through the nasal septum such that the nasal septum is heated above about 50° C. and up to about 57° C. to allow reformation thereof, and wherein the cooling fluid is circulated within the lumen to maintain tissue in immediate contact with the electrode in a cooled state.

12. The septoplasty instrument according to claim 11, wherein the cooling fluid is one of water and glycol.

13. The septoplasty instrument according to claim 11, wherein a first electrical potential is provided to the first electrode member and wherein a second electrical potential is provided to the second electrode member such that energy is conducted between the electrodes and through the nasal septum disposed therebetween.

14. The septoplasty instrument according to claim 11, further comprising a ratchet mechanism configured for selectively locking the shaft members relative to one another.

15. The septoplasty instrument according to claim 11, wherein the cooling fluid circulating through the lumen of the at least one electrode members maintains the tissue surrounding the electrode member at below about 40° C.

16. A method of performing a septoplasty, comprising the steps of:
providing first and second electrode members, each electrode member including an electrode disposed within an insulative housing, the housing of at least one electrode including a lumen defined therein configured to circulate a cooling fluid therethrough, the lumen extending a length of the electrode member along an outer periphery thereof, at least one of the electrodes adapted to connect to a source of electrosurgical energy for conducting energy through tissue disposed therebetween, and wherein, upon application of electrosurgical energy to the at least one electrode, energy is conducted between the electrodes and through the nasal septum such that the nasal septum is heated;
inserting the first electrode member into a first nostril of a patient;
inserting the second electrode member into a second nostril of the patient such that the nasal septum is grasped between the first and second electrode members;
conducting electrosurgical energy between the first and second electrode members and through the nasal septum to heat the nasal septum above about 50° C. and up to about 57° C. to allow reformation thereof while circulating the cooling fluid within the lumen to maintain tissue surrounding the electrode in a cooled state; and
reforming the nasal septum to a reformed configuration.

17. The method according to claim 16, further comprising the steps of:
maintaining the nasal septum in the reformed configuration; and
allowing the nasal septum to cool such that the nasal septum retains the reformed configuration.

18. The method according to claim 16, wherein the cooling fluid circulating through the lumen of the at least one electrode members maintains the tissue surrounding the electrode member below about 40° C.

19. The method according to claim 16, wherein the first and second electrode members are disposed at distal ends of respective first and second shaft members, the first and second shaft members moveable with respect to one another about a pivot to move the electrode members between a spaced position and a closer position for grasping the nasal septum therebetween.

20. A septoplasty instrument, comprising:
a pair of electrodes configured for positioning on either side of the septum, at least one of the electrodes adapted to connect to a source of electrosurgical energy for conducting electrosurgical energy between the electrodes and through the septum to heat the septum; and
a monitoring sensor configured to monitor a relative increase in conductivity of the septum during heating, wherein,
the electrosurgical energy supplied to the at least one electrode is controlled in accordance with the relative increase in conductivity of the septum such that a target relative increase in electrical conductivity is attained during heating of the septum.

21. The septoplasty instrument according to claim 20, wherein the target relative increase in conductivity of the septum is about 40%.

22. The septoplasty instrument according to claim 20, wherein the target relative increase in conductivity is maintained via a closed loop control.

23. The septoplasty instrument according to claim 20, wherein at least one of the electrodes includes a housing having a lumen defined therein and configured to circulate a cooling fluid therethrough for maintaining tissue surrounding the electrode in a cooled state during heating of the septum.
